(19) United States
(12) Patent Application Publication
(10) Pub. No.: US 2004/0199048 A1
(43) Pub. Date: Oct. 7, 2004

(54) High flow stone basket system

(76) Inventors: Ralph V Clayman, Irvine, CA (US); Nabil Hilal, Laguna Niguel, CA (US); Edward D Pingleton, Laguna Niguel, CA (US); Richard C Ewers, Fullerton, CA (US)

Correspondence Address: APPLIED MEDICAL RESOUCES CORPORATION, 22872 Avenida Empresa, Rancho Santa Margarita, CA, 92688, US

(21) Appl. No.: 10/492,267

(22) PCT Filed: Jun. 14, 2002
(86) PCT No.: PCT/US02/18975

Related U.S. Application Data
(60) Provisional application No. 60/329,241, filed on Oct. 12, 2001.

(51) Int. Cl. A61B 1/00

(52) U.S. Cl. 600/104; 600/156

(57) ABSTRACT

A medical device for viewing inside a body and for retrieving an object from a location within the body, the medical device comprising an endoscope having a tube defining a working channel and a basket engageable with the object. In one embodiment of the invention, the basket does not require a sheath and is disposed through the working channel such that the endoscope operates to contain or activate the sheathless basket. In another embodiment of the invention, a single push wire with a collar at its end is used in place of a sheath to control the opening and closing of the basket. In another embodiment of the invention, a tapered and funnel-shaped sheath surrounding the basket is used to contain or activate the basket. The invention minimizes the basket crossing profile so as to improve fluid flow in the working channel.

### CROSS REFERENCE TO RELATED APPLICATIONS

This application claims benefit to U.S. Provisional Application Serial No. 60/329,241, filed Oct. 12, 2001, entitled “High-Flow Stone Basket System” as well as PCT Application Serial No. PCT/US02/18975 filed Jun. 14, 2002, entitled “High-Flow Stone Basket System”, both of which are incorporated herein by reference.

### BACKGROUND OF THE INVENTION

1. Field of the Invention

The present invention generally relates to medical devices and, more particularly, to a medical device for endoscopic retrieval of an object such as a stone from the body.

2. Description of Related Art

Endoscopic urological procedures treat pathologies in the urinary system in a minimally invasive way by taking advantage of naturally existing body passages. These passages, such as the urethra and the ureter, allow a surgeon to reach deep inside the body with specially constructed instrumentation and tools. Endoscopic instrumentation and tools are manipulated from outside the body and can perform various functions including retrieval of kidney stones. Endoscopic removal of kidney stones is performed under visualization provided by a urological endoscope or ureteroscope. The scopes, which may be rigid or flexible, allow visualization of the urinary system from the inside. The scopes typically feature an open lumen or “working channel” extending from end-to-end. The working channel of the scope can accommodate specialized tools designed to retrieve urinary stones. One version of these tools is called a “stone basket”.

Stone baskets slidably fit in the working channels of the scopes. Typical baskets have crossing profiles of approximately 2.5 Fr to 3 Fr, which occupy most of the working channel in a scope. Specifically, a conventional basket includes an outer sleeve (e.g., a plastic tube) that functions to open and close the basket by sliding over or off of the basket wires. The working channel is also used to flush saline through the tip of the scope, which cleanses the scope for clear endoscopic visualization and distends the surrounding tissue so as to create an open working field. Thus, fluid flow and visualization become poor when a scope has its working channel almost completely filled with the basket and the outer sleeve.

Accordingly, there is a need in the art for a medical device for endoscopic retrieval of an object from the body that provides improved fluid flow.

### SUMMARY OF THE INVENTION

A medical device for viewing inside a body and for retrieving an object from a location within the body, the medical device comprises an endoscope having a tube defining a working channel and a sheathless basket engageable with the object. The sheathless basket is disposed through the working channel such that the endoscope operates to contain or activate the sheathless basket. The sheathless basket facilitates fluid flow through the working channel by not requiring an outer sleeve. The endoscope, rather than the outer sleeve, serves to activate or deactivate the basket.

In another embodiment of the invention, a single push wire with a collar at its end is used to control the opening and closing of a basket in place of a sheath or outer sleeve. In yet another embodiment of the invention, a tapered and funnel-shaped sheath surrounding a basket is used so as to minimize the basket crossing profile and to improve fluid flow. The sheath may include a ribbed extrusion providing strength to guide the basket wires, and channels running along the surface to guide fluid flow. The sheath may also be slotted along most of the length of the basket wires to remove material and to create flow channels.

Other features of the present invention include basket configurations that allow close stone proximity for easy retrieval, and wire construction that offers flexibility for full scope deflection.

### DESCRIPTION OF THE DRAWINGS

The accompanying drawings, which are incorporated in and constitute a part of this specification, illustrate the embodiments of the invention and, together with the description, explain the features, advantages and principles of the invention. In the drawings:

FIG. 1 illustrates a common basket system of the prior art;

FIG. 2 illustrates a deactivated basket device in a basket system of the prior art;

FIG. 3 illustrates an activated basket device in a basket system of the prior art;

FIG. 4 illustrates a basket system in accordance with a first embodiment of the invention;

FIG. 5 illustrates a deactivated basket device of the basket system in accordance with the first embodiment of the invention;

FIG. 6 illustrates an activated basket device of the basket system in accordance with the first embodiment of the invention;

FIG. 7 illustrates a sheathless basket device of a basket system in accordance with the first embodiment of the invention;

FIG. 8 is a perspective view of a sheathed basket device in accordance with a second embodiment of the invention;

FIG. 9 is a side view of the sheathed basket device in accordance with the second embodiment of the invention;

FIG. 10 illustrates a basket system in accordance with the second embodiment of the invention;

FIG. 11 illustrates a basket device in accordance with a third embodiment of the invention;

FIG. 12 illustrates a basket system in accordance with a fourth embodiment of the invention;

FIG. 13 illustrates a basket sub-assembly of the basket system as shown in FIG. 12;

FIG. 14 illustrates a basket configuration in accordance with an embodiment of the invention;

FIG. 15 is a top plan view of a basket tip in accordance with the basket configuration of FIG. 14;

FIG. 16 is a top plan view of a basket tip in accordance with a second basket configuration;

FIG. 17 is a top plan view of a basket tip in accordance with a third basket configuration;

FIG. 18 is a side view of another basket configuration in accordance with an embodiment of the invention; and

FIG. 19 is a perspective view of the basket configuration of FIG. 18.

### DESCRIPTION OF PREFERRED EMBODIMENTS AND BEST MODE OF THE INVENTION

The following detailed description refers to the accompanying drawings that illustrate the embodiments of the present invention. Other embodiments are possible and modifications may be made to the embodiments without departing from the spirit and scope of the invention. Thus, the following detailed description is not meant to limit the invention. Rather the scope of the invention is defined by the appended claims.

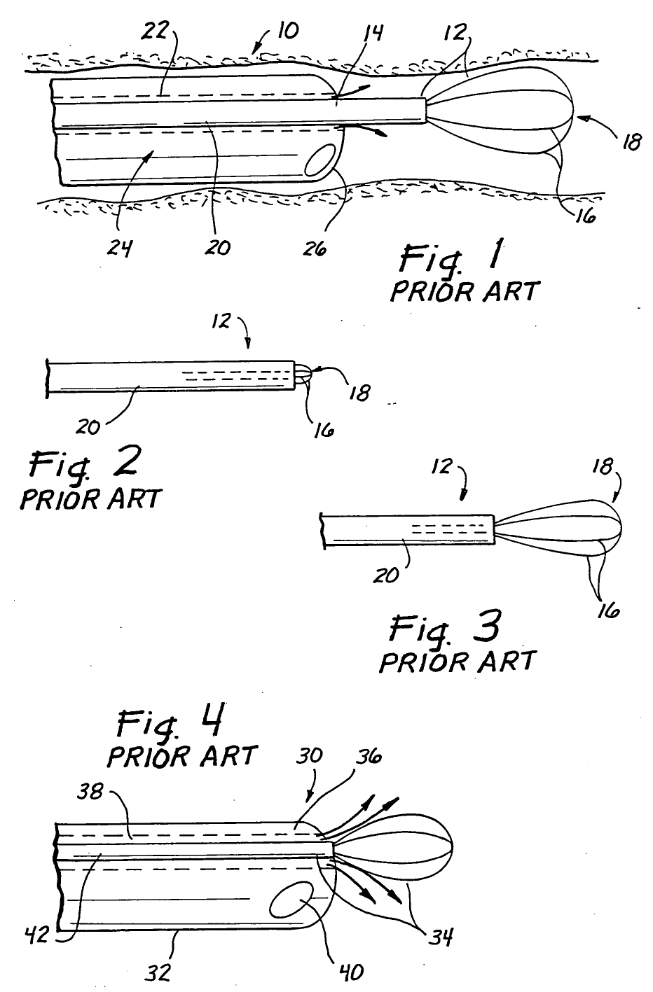

FIG. 1 illustrates a common basket system 10 of the prior art. The basket system 10 comprises a basket device 12, a basket sheath 20 and an endoscope 24. Basket device 12 and basket sheath 20 are disposed in a working channel 22 of endoscope 24. Basket device 12 includes a rod 14 and a plurality of looped wires 16 collectively forming a basket 18. Basket device 12 is disposed in and through basket sheath 20 as shown in FIGS. 2 and 3. Basket sheath 20 surrounds rod 14 of basket device 12 and serves as a means to contain or activate basket 18. In particular, basket 18 may be activated to affect a high profile basket device 12 as shown in FIG. 3, or deactivated to form a low profile basket device 12 as shown in FIG. 2.

To activate basket 18, basket device 12 is advanced distally with respect to basket sheath 20 such that looped wires 16 are no longer constrained by the lumen of basket sheath 20. To deactivate basket 18, basket device 12 is retracted or moved proximally with respect to basket sheath 20 as shown in FIG. 2. Looped wires 16 are withdrawn and contained within the lumen of basket sheath 20 to affect the collapsed or low profile state of basket 18. A drawback of conventional stone basket systems is that basket sheath 20 occupies much of the space in working channel 22, thereby limiting the cross-sectional area available for fluid flow and irrigation. That is, fluid flow and irrigation are restricted due to the presence of basket sheath 20.

FIGS. 4-7 illustrate a basket system 30 according to a first embodiment of the invention. Basket system 30 is preferably adapted to retrieve objects from a location inside a body such as stones and fragmentations while providing endoscopic visualization. In the first embodiment of the invention, basket system 30 comprises an endoscope 32 and a basket device 34. Endoscope 32 has a tube 36 defining a lumen or working channel 38. Endoscope 32 may also include optical components 40. Referring to FIG. 7, basket device 34 comprises an elongate member 42 and a plurality of looped wires 44 coupled to a distal end 46 of elongate member 42. Looped wires 44 form a basket 48. The number of looped wires 44 is based on a particular configuration of basket 48 desired for a specific purpose.

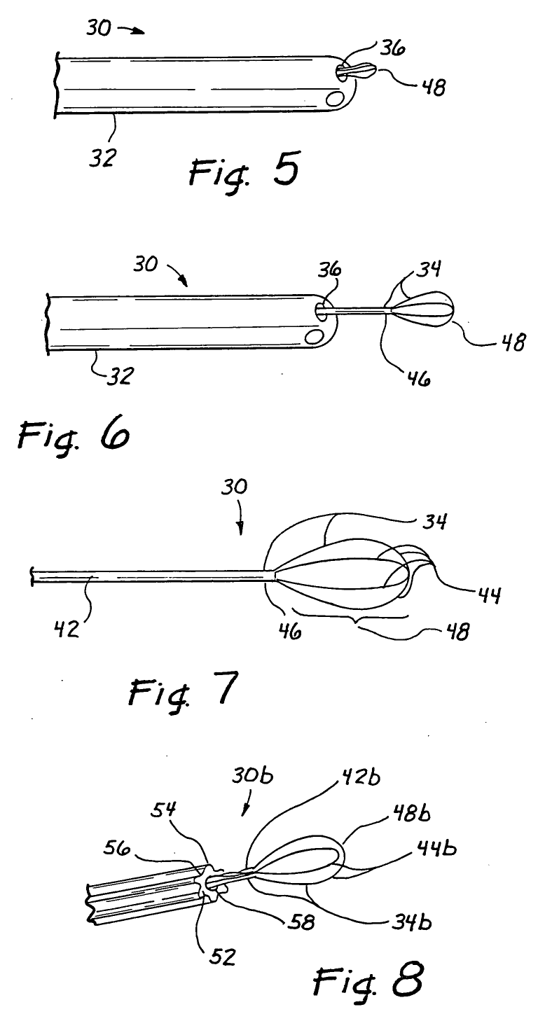

As shown in FIGS. 4-7, it will be appreciated that the conventional basket sheath has been omitted from basket system 30, thereby maximizing fluid flow as greater cross-sectional area is provided in basket system 30. In the sheathless basket system 30, endoscope 32 serves to contain and activate basket 48. Basket device 34 is disposed through working channel 38 and is axially movable with respect to endoscope 32. To contain or deactivate basket 48, elongate member 42 is pulled axially in a proximal direction with respect to endoscope 32 which causes basket 48 to collapse within working channel 38. In particular, basket 48 collapses into a low profile state and is enclosed within working channel 38 as illustrated in FIG. 5.

To activate basket 48, basket device 34 is moved in a distal direction with respect to endoscope 32 as shown in FIG. 6. No longer bound by working channel 38, looped wires 44 expand into a high profile state of basket 48. It will be appreciated that endoscope 32 itself serves as the means to activate and deactivate basket 48. Without a sheath or outer sleeve, basket device 34 provides a flow rate higher than what would be possible with conventional basket systems needing basket sheaths. Specifically, the fluid passageway defined by the area between the external surface of elongate member 42 and the inner surface of working channel 38 is increased.

In the sheathless system shown in FIGS. 4-7, basket device 34 is adapted to be operated with endoscope 32 and positioning of basket 48 depends upon the movement and placement of tube 36. Although basket device 34 is adapted to protrude distally from tube 36, basket device 34 can only extend a certain distance axially from tube 36 since elongate member 42 is generally thin and flexible.

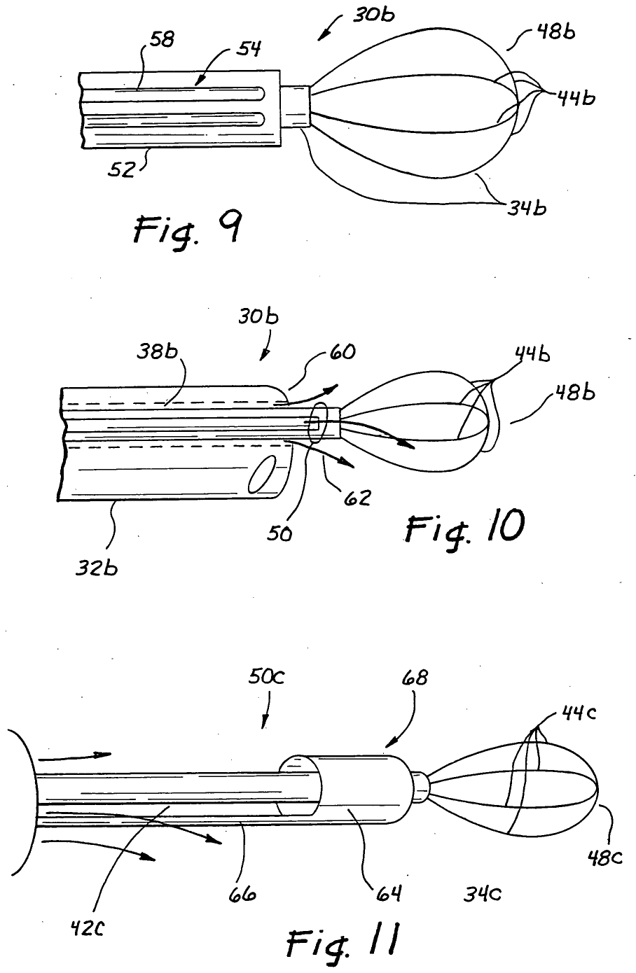

FIGS. 8-10 illustrate a sheathed basket device 30b according to a second embodiment of the invention. Sheathed basket device 30b includes a basket sub-assembly 50 (see FIG. 10) that enables basket operation independent of endoscope 32b while optimizing fluid flow in endoscopic working channel 38b. Basket sub-assembly 50 comprises a ribbed basket sheath 52 having alternating configuration of axial ribs 54 and grooves/channels 56. Basket sheath 52 is inserted through a proximal end of endoscope 32b and is adapted to extend through the length of working channel 38b.

Basket sub-assembly 50 further includes a basket device 34b that is disposed in and through basket sheath 52. Similarly to basket device 34 in FIGS. 4-7, basket 34b comprises an elongate member 42b and a plurality of looped wires 44b forming a basket 48b. Basket 48b is deactivated by pulling elongate member 42b in a proximal direction causing looped wires 44b to collapse within a lumen 58 of basket sheath 52. With basket 48b collapsed and enclosed within lumen 58, basket device 34b is secured to basket sheath 52 and sub-assembly 50 may be moved as one structure with respect to endoscope 32b. With this embodiment of the invention, basket sub-assembly 50 may be moved distally beyond endoscope tip 60 for an extended distance inside a body so as to position basket sub-assembly 50 adjacent to an object, e.g., a stone, to be retrieved. To activate basket 48b, basket device 34b is moved distally with respect to basket sheath 52 to a desired location and basket device 34b is advanced so as to expand looped wires 44b into a high profile state.

The sheathed basket device 30b enables operation of basket sub-assembly 50 independent of endoscope 32b while facilitating improved fluid flow. In particular, ribbed basket sheath 52 enhances fluid flow by providing grooves/channels 56 that direct fluid flowing in endoscopic working channel 38b.

FIG. 11 illustrates a basket sub-assembly 50c according to a third embodiment of the invention. Basket sub-assembly 50c comprises a basket actuator 64 and a basket device 34c. Basket device 34c comprises an elongate member 42c coupled to a plurality of looped wires 44c, which form a basket 48c. Basket actuator 64 comprises a thin control rod 66, which extends through the endoscopic working channel of the basket system (not shown), and a closure collar 68 coupling to a distal end of control rod 66. Basket actuator 64 is adapted to releasably retain basket 48c.

To deactivate basket 48c, elongate member 42c is pulled in a proximal direction to cause looped wires 44c to collapse within closure collar 68. With basket 48c collapsed and enclosed within closure collar 68, basket device 34c is secured to basket actuator 64 and basket sub-assembly 50c may be moved as one structure with respect to the endoscope. Therefore, basket sub-assembly 50c may be moved distally beyond the endoscope tip for an extended distance inside a body so as to position basket sub-assembly 50c adjacent to an object to be retrieved. To activate basket 48c, basket device 34c is moved distally with respect to closure collar 68 to a desired location and is advanced so as to expand looped wires 44c into a high profile state.

It will be appreciated that basket actuator 64 facilitates high fluid flow in the endoscope since control rod 66, which extends through the endoscopic working channel, occupies minimal cross-sectional area. In particular, control rod 66 does not wrap or surround elongate member 42c as would a conventional sheath. As a result, the cross-sectional area of the fluid passageway is increased, thereby increasing fluid flow in the endoscope.

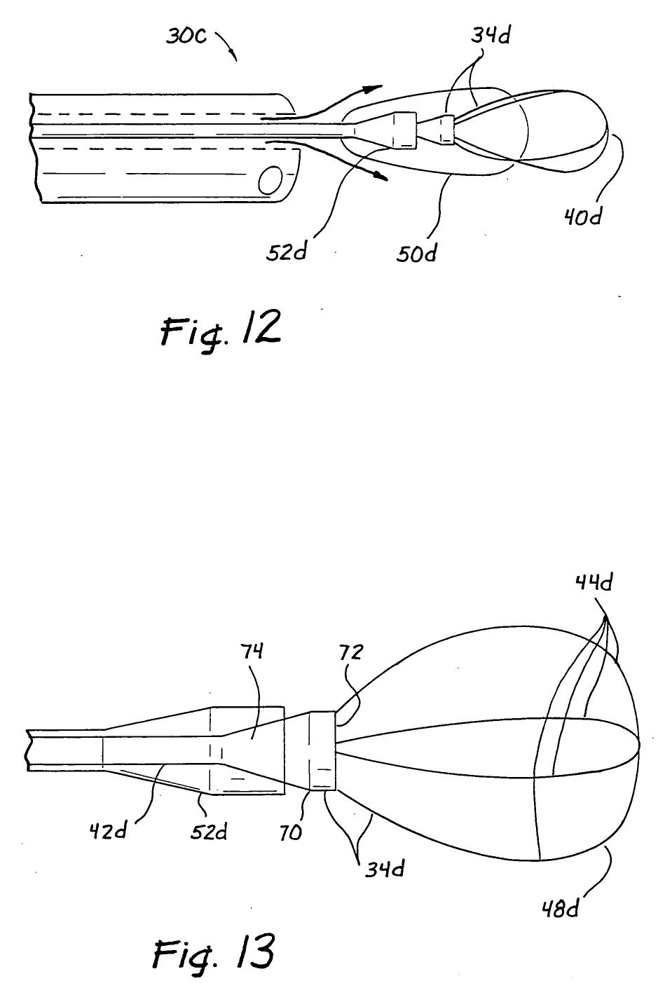

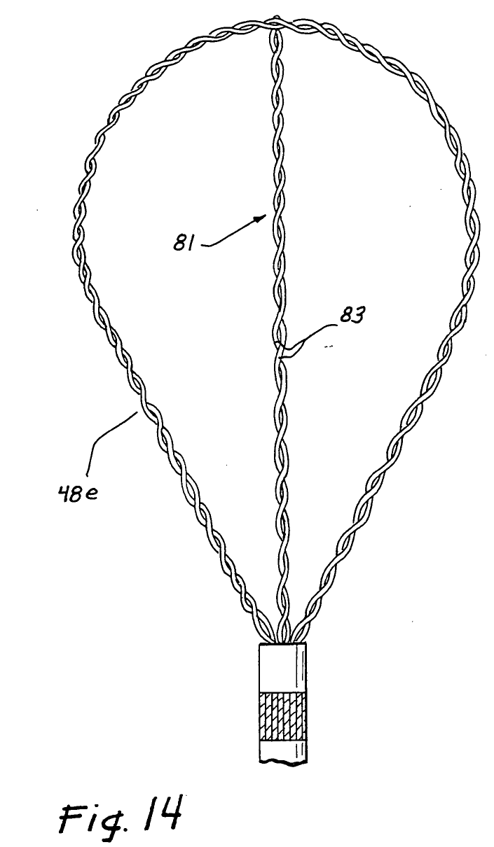

FIGS. 12 and 13 illustrate a basket system 30c according to a fourth embodiment of the invention. Basket system 30c includes a basket sub-assembly 50d, which includes a basket device 34d, a tapered elongate member 42d and a tapered basket sheath 52d. Elongate member 42d comprises a distal portion 70 that increases in diameter as it extends distally so as to provide a distal end 72 with an expanded cross-sectional area for coupling a plurality of looped wires 44d, which form a basket 48d. The increased cross-sectional area of distal end 72 facilitates a stronger bond with looped wires 44d as more surface area is provided for coupling. To accommodate tapered elongate member 42d without limiting fluid flow, tapered basket sheath 52d is provided. Similarly to elongate member 42d, basket sheath 52d comprises a distal portion 74 that increases in diameter as it extends distally. That is, sheath distal portion 74 corresponds and conforms to the outer surface of distal portion 70 of elongate member 42d. The remainder of basket sheath 52d proximal to distal portion 74 has a smaller and uniform diameter that is adapted to occupy minimal space within the endoscope working channel. As a result, basket sheath 52d facilitates high fluid flow while serving as an actuator for basket 48d.

To deactivate basket 48d, elongate member 42d is pulled in a proximal direction to cause looped wires 44d to collapse within the distal portion 74 of basket sheath 52d. The tapered characteristic of distal portion 74 enables it to receive tapered distal portion 70 of elongate member 42d while enclosing looped wires 44d in a low profile state. Basket device 34d is thus secured to basket sheath 52d and sub-assembly 50d may then be moved as one structure. With this embodiment, basket sub-assembly 50d may be moved distally beyond the endoscope tip for an extended distance inside a body so as to position basket sub-assembly 50d adjacent to an object to be retrieved. Basket device 34d may then be moved distally with respect to distal portion 74 of basket sheath 52d to activate basket 48d.

In each of the above basket systems, it will be appreciated that better endoscope deflection is facilitated by minimizing the material disposed in the working channel of the endoscope, which also provides better resistance to bending and kinking. This is accomplished by eliminating the basket sheath altogether, which results in a naked basket device wherein deflection would only be resisted by the elongate member of the basket device, or by minimizing the space and material of the basket sheath by using a sheath with a lesser thickness or using a control rod.

As to the wires used in the invention, a 0.030″ to 0.034″ nitinol wire is used with three wire loops of much smaller diameter attached around the nitinol wire. The looped wires are placed loops first into the working channel of the endoscope. Upon entry, the loops collapse either manually or by means of a basket sheath. Once placed through the scope, the loops may be extended past the tip of the scope. There, freed from the constraints of the working channel, the loops will expand automatically. With the wire loops placed over an object to be retrieved such as a stone, the loops are retracted back into the scope working channel by pulling on the proximal end of the tool that is outside the patient. The user stops pulling when the stone butts up against the leading edge of the scope. The scope and stone may then be extracted from the patient together.

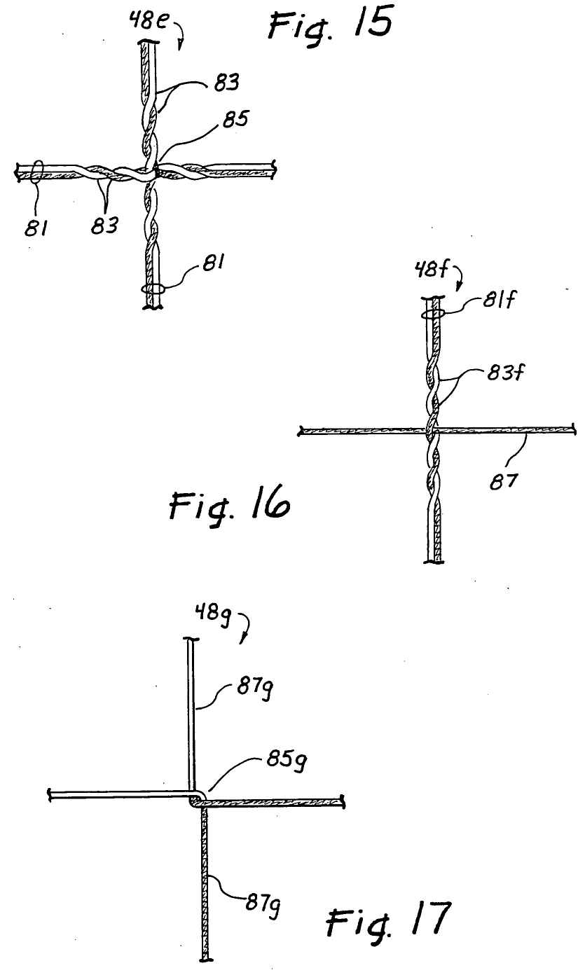

FIG. 14 illustrates a basket configuration 48e in accordance with an embodiment of the invention. Basket 48e can be used in any one of the above mentioned basket devices of the invention. Basket 48e includes a plurality of cords 81, each of cords 81 includes a pair of intertwined, helical strands 83. The strands 83 may comprise nitinol wires. In one method of assembling the cords, a first pair of untwisted strands 83 is intertwined with a second pair of untwisted strands 83. The strands 83 are then twisted thereby locking the strands of the first pair with the strands of the second pair at a basket tip 85 (see top plan view of FIG. 15). It will be noted that the double helix configuration also provides each cord 81 with greater rigidity, enabling basket 48e to hold a high profile shape when released from a scope or basket sheath. It should be noted that each cord 81 may comprise a single strand 83 doubled back on itself providing two juxtaposed strand portions that are then twisted forming the double helix configuration.

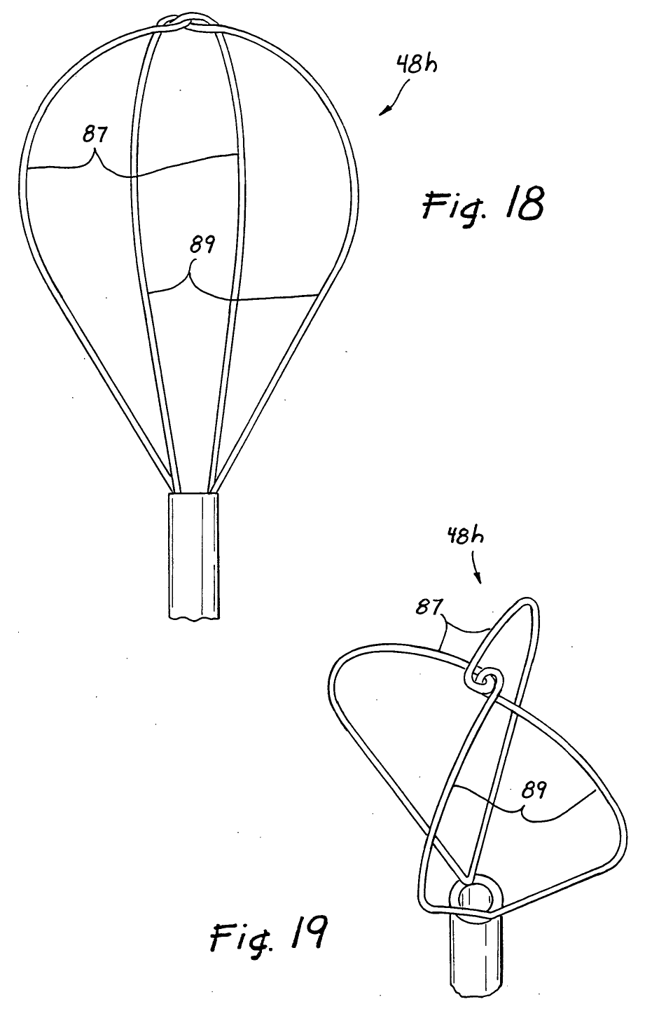

FIGS. 16 and 17 illustrate alternative basket configurations that obviate the need for a loop. FIG. 16 is a top plan view of an alternative embodiment of a basket 48f. As shown in FIG. 16, a single wire 87 may be inserted through a double helix cord 81f comprising a pair of intertwined strands 83f. FIG. 17 is a top plan view of another embodiment of a basket 48g. As shown in FIG. 17, basket 48g comprises two untwisted wires 87g intertwined at a basket tip 85g.

FIGS. 18 and 19 illustrate another basket configuration 48h of the invention. Basket 48h comprises a first perpendicularly looped wire 87 intertwined with a second perpendicularly looped wire 89. In contrast to the prior art where each wire is disposed on a single plane, each of wires 87 and 89 of the invention extends along two perpendicular planes.

Many alterations and modifications may be made by those having ordinary skill in the art without departing from the spirit and scope of the invention. Therefore, it must be understood that the illustrated embodiments have been set forth only for the purposes of examples and that they should not be taken as limiting the invention.

1. A medical device for viewing inside a body and for retrieving an object from a location within the body, comprising:
an endoscope having a tube defining a working channel; and
a sheathless basket engageable with the object, said sheathless basket being disposed through the working channel,
wherein the endoscope operates to contain or activate the sheathless basket,
wherein the sheathless basket facilitates fluid flow through the working channel.

2. The medical device of claim 1, wherein the sheathless basket moves axially with respect to the endoscope.

3. The medical device of claim 1, wherein the sheathless basket comprises an elongate member and a plurality of wires coupled to a distal end of the elongate member.

4. The medical device of claim 3, wherein the sheathless basket is retracted in the endoscope by axially pulling the elongate member in a proximal direction with respect to the endoscope.

5. The medical device of claim 3, wherein the sheathless basket is activated by axially pushing the elongate member in a distal direction with respect to the endoscope.

6. The medical device of claim 5, wherein the looped wires expand when the sheathless basket is activated.

7. The medical device of claim 2, wherein positioning of the sheathless basket is achieved by moving the endoscope to a desired location.

8. The medical device of claim 1, wherein the endoscope is flexible.

9. The medical device of claim 8, wherein the endoscope can be deflected in a “U” or a “J” shape.

10. The medical device of claim 3, wherein the sheathless basket facilitates fluid flow through the working channel by increasing an area between an external surface of the elongate member and an inner surface of the tube.

11. A medical device for viewing inside a body and for retrieving an object from a location within the body, comprising:
an endoscope having a tip and a tube defining a working channel;
a ribbed basket sheath inserted in the tube of the endoscope extending through the length of the working channel, said ribbed basket sheath defining a lumen; and
a basket device engageable with the object, said basket device being disposed in the lumen of the ribbed basket sheath,
wherein the ribbed basket sheath facilitates fluid flow through the working channel.

12. The medical device of claim 11, wherein the ribbed basket sheath includes a plurality of axial grooves and a plurality of axial ribs.

13. The medical device of claim 11, wherein the ribbed basket sheath includes a plurality of channels.

14. The medical device of claim 11, wherein the basket device comprises an elongate member and a plurality of cords coupled to a distal end of the elongate member.

15. The medical device of claim 14, wherein each cord comprises a pair of intertwined, helical strands.

16. The medical device of claim 15, wherein a first pair of strands of a first cord is twisted with a second pair of strands of a second cord at a basket tip so as to form a double helix configuration securing the first cord with the second cord.

17. The medical device of claim 16, wherein the double helix configuration provides rigidity to the cords enabling the basket device to expand into a high-profile shape when released from the ribbed basket sheath.

18. The medical device of claim 14, wherein each of a first cord and a second cord comprises a single strand doubled back on itself, wherein the first cord and the second cord are juxtaposed and twisted forming a double helix configuration.

19. The medical device of claim 14, wherein the cords comprise a single wire and a pair of intertwined strands forming a double helix cord, wherein the single wire is inserted through an opening of the double helix cord.

20. The medical device of claim 14, wherein the cords comprise two untwisted wires intertwined at a basket tip.

21. The medical device of claim 14, wherein the cords comprise a first perpendicularly looped wire intertwined with a second perpendicularly looped wire.

22. The medical device of claim 14, wherein the basket device is deactivated by axially pulling the elongate member in a proximal direction with respect to the ribbed basket sheath.

23. The medical device of claim 22, wherein the basket device is retracted and enclosed within the lumen of the ribbed basket sheath.

24. The medical device of claim 23, wherein the basket device and the ribbed basket sheath move together as a single structure with respect to the endoscope.

25. The medical device of claim 23, wherein the basket device and the ribbed basket sheath can be moved distally beyond the endoscope tip so as to position the basket device adjacent to the object to be retrieved.

26. The medical device of claim 14, wherein the basket device can be positioned at the location within the body for retrieval of the object by axially guiding the ribbed basket sheath to that location.

27. The medical device of claim 26, wherein the basket device is activated by axially pushing the elongate member in a distal direction with respect to the ribbed basket sheath.

28. A medical device for viewing inside a body and for retrieving an object within the body, comprising:
an endoscope having a tip and a tube defining a working channel;
a basket actuator including a control rod and a closure collar coupled to a distal end of the control rod, said basket actuator extending through the length of the working channel; and
a basket device comprising an elongate member having a proximal end and a distal end and a plurality of looped wires coupled to the distal end of said elongate member, said basket device is disposed through the basket actuator,
wherein the closure collar is adapted to activate or deactivate the basket,
wherein the control rod has a first cross-sectional area and the closure collar has a second cross-sectional area,
wherein the first cross-sectional area is smaller than the second cross-sectional area so as to facilitate fluid flow through the working channel.

29. The medical device of claim 28, wherein the basket device is deactivated by axially pulling the elongate member in a proximal direction so as to retract and secure the looped wires within the closure collar.

30. The medical device of claim 29, wherein the basket device and the basket actuator move together as a single structure with respect to the endoscope.

31. The medical device of claim 29, wherein the basket actuator can be moved distally beyond the endoscope tip so as to position the basket device adjacent to the object to be retrieved.

32. The medical device of claim 28, wherein the basket device can be positioned at the location within the body to retrieve of the object by axially guiding the control rod of the basket actuator to that location.

33. The medical device of claim 32, wherein the basket device is activated by axially pushing the elongate member in a distal direction away from the closure collar.

34. The medical device of claim 28, wherein the control rod facilitates deflection of the basket actuator.

35. A medical device for viewing inside a body and for retrieving an object from a location within the body, comprising:
an endoscope having a tip and a tube defining a working channel;
a tapered basket sheath having a proximal portion, a distal portion and a cross-sectional area, wherein the cross-sectional area of the basket sheath increases from the proximal portion to the distal portion, wherein said basket sheath is inserted in the tube of the endoscope extending through the length of the working channel; and
a basket device engageable with the object, said basket device comprising an elongate member having a proximal portion, a distal portion and a cross-sectional area, wherein the cross-sectional area of the elongate member increases from the proximal portion to the distal portion, said basket device further comprising a plurality of looped wires coupled to the distal portion, wherein said basket device is disposed in the basket sheath such that the sheath distal portion conforms to an outer surface of the distal portion of the elongate member.

36. A medical device for retrieving an object from a location within a body, comprising a slidable basket engageable with the object, wherein the basket comprises:
a plurality of cords, each cord comprising a pair of intertwined, helical strands,
wherein a first pair of strands of a first cord is intertwined with a second pair of strands of a second cord at a basket tip so as to form a double helix configuration securing the first cord with the second cord.

37. The medical device of claim 36, wherein the strands comprise nitinol wires.

38. The medical device of claim 37, wherein the double helix configuration provides a secure coupling of the cords.
